(19) United States
(12) Patent Application Publication
(10) Pub. No.: US 2014/0141095 A1
(43) Pub. Date: May 22, 2014

(54) USE OF DEUTERIUM DEPLETED WATER FOR THE TREATMENT OF INSULIN RESISTANCE

(71) Applicant: Gábor Somlyai, Budapest (HU)

(72) Inventor: Gábor Somlyai, Budapest (HU)

(21) Appl. No.: 13/808,074

(22) PCT Filed: Jul. 8, 2011
(86) PCT No.: PCT/HU2011/000063

§ 371 Date: Jan. 24, 2013

(30) Foreign Application Priority Data
Jul. 8, 2010 (HU) P1000357

(51) Int. Cl. A61K 33/00; A61K 31/70; A61K 38/00

(52) U.S. Cl. CPC A61K 33/00; A61K 38/00; A61K 31/70
USPC 424/600; 514/6.8; 423/580.1

(57) ABSTRACT

The invention relates deuterium depleted water containing 0.01 to 135 ppm deuterium, preferably 105 to 125 ppm deuterium, for use in the treatment of insulin resistance.

Further object of the invention is deuterium depleted food product containing 0.01 to 135 ppm deuterium, preferably 105 to 125 ppm deuterium, for use in the treatment of insulin resistance.

The invention relates to pharmaceutical and food products containing deuterium depleted water, suitable for the treatment of insulin resistance.

### TECHNICAL BACKGROUND

Diabetes mellitus (DM) is a chronic disease of human metabolism. The cause of disease is lack of insulin, a hormone produced by the pancreas, or the insensitivity to insulin within the organism (insulin resistance, relative lack of insulin), or both. Due to the absolute or relative lack of insulin the cells are unable to take up glucose, that is why the blood glucose level increases (hyperglycemia) leading to the symptoms of the disease. The patients' blood glucose level is in such cases higher than the normal range of 3.5-6.5 mmol/L. It is common for persons in this disease group that, in spite of different pathogenesis, they cannot secrete the amount of insulin required by their metabolic processes, or insulin, although present, has no effect.

From diagnostic aspect, diabetes mellitus (DM) has three subgroups:

1. Insulin-dependent, type I DM (IDDM) develops typically in children and young adults but can occur at any age. In patients belonging to this group, endogenous insulin is fully absent, making administration of insulin inevitable for reducing blood sugar and for mere survival.

2. Non-insulin-dependent, type II DM (NIDDM) usually develops above 30 years of age. The patients are typically obese and insulin resistant; that is, their organism produces insulin but the normal insulin amount induces in them a subnormal response, so blood glucose level increases. They do not need external insulin for survival.

3. Other cases of DM, not fitting into either group, are caused by another primary disease, e.g., that of the pancreas.

In the population of a country, diabetics can make up 2 to 12%. Within the diabetic subpopulation, type I occurs in ca. 15%, and type II in ca. 85%. Based on the geographic distribution of DM is likely that high prevalence is partly connected to rich nutrition and obesity (in China, the prevalence of NIDDM is 1.3%; in the USA, 6.6%; and among the Mexicans in the USA, 16%). The causal factors of the disease are mostly known, and its treatment can be regarded as solved in some sense, though medication over several decades may have serious side effects. Diabetics can show “acute” complications—hypoglycemia, hyperosmotic coma, lactic acid acidosis etc.—any time, and over the years late complications can develop (macroangiopathy, indicated by higher incidence of atherosclerotic complications; microangiopathy, a special damage of capillaries; or increased infection susceptibility). All that explains that diabetics have 2-3 times higher mortality, 10 times higher occurrence of blindness, and 20 times higher occurrence of gangrene and amputation of extremities, than the healthy population. The diabetic population has extremely high “social” costs (e.g. they have twice more hospital stays than the average population), which further emphasizes the need and request of the society for more efficient treatment of DM.

The aim of DM treatment is to avoid the direct consequences of lack of insulin and to reduce the complications of the chronic state.

The treatment of DM can be optimized through three factors: 1. Modification of diet; 2. Careful planning of physical activity; 3. Medication. Ideally, well-planned food intake, sugar utilization by physical activity and exactly dosed medicines are in harmony, and provide for appropriate and balanced blood glucose level of the organism with minimal deviations. Unfortunately, this can be hardly achieved and kept for longer periods, which leads to the complications described above.

Recently, papers were published (FEBS Lett. 1993; 317: 1-4, Természetgyógyászat 1996; 10: 29-32, Kisállatorvoslás 1996; 3:114-5, Erfahrungsheilkunde 1997; 7: 381-88, J. R. Heys and D. G. Melillo (eds) 1997 Synthesis and Applications of Isotopically Labelled Compounds. John Wiley and Sons Ltd. pp. 137-141, Z. Onkol/J. of Oncol. 1998; 30: 91-94), and Hungarian patents were registered (Reg. No.: 208084, 209787), based on the new knowledge on the important role of naturally occurring deuterium (D) in the regulation of cell division. Beyond the animal experiments described in the patents, the anti-tumor effect of deuterium-depleted water (DDW) has been verified since also in human studies. In the last ten plus years, nearly 10 thousand patients consumed ca. 2000 tons of DDW, and 1500 cancer patients were followed-up for long time. These cases confirmed that the cancer cells are sensitive to D depletion and in the majority of cases (70-80%) cannot adapt to the altered environment, resulting in shrinkage or even total elimination of the tumor.

There was a number of diabetic persons among the tumor patients who consumed DDW in the last years. The patients began to drink DDW because of their cancer but, surprisingly, it was beneficial also for their diabetes. We could draw the unexpected consequence that lowering D concentration has advantageous influence also on the blood glucose level in a diabetic organism. In numerous cases the patients consuming DDW could reduce their dose of insulin, or other medicines used to prevent permanent hypoglycemia, which indicated that D depletion increased the biological efficiency of insulin. Based on this observation, a patent application was submitted and received the European patent No. 1465641. However, the description of that patent does not mention that DDW is suitable for treatment of insulin resistance, or that water with 105-125 ppm D-level is optimal for that effect (see below).

### SUMMARY OF THE INVENTION

The invention relates primarily to deuterium depleted water with 0.01-135 ppm deuterium content for use in the treatment of insulin resistance.

Alternatively, the invention relates to the use of deuterium depleted water with 0.01-135 ppm deuterium content in manufacturing of pharmaceutical products applicable in the treatment of insulin resistance.

The invention further relates to deuterium-depleted food products (with 0.01-135 ppm deuterium content) for use in the treatment of insulin resistance.

Alternatively: use of deuterium depleted water with 0.01-135 ppm deuterium content in the manufacturing of food products applicable in the treatment of insulin resistance.

Such above-described food products or their use is preferred if the food product contains carbohydrates, proteins or lipids with 0.01-135 ppm, optimally 105-125 ppm deuterium content.

A further aspect is a method for the treatment of insulin resistance in which a person requiring treatment is administered with deuterium-depleted water (DDW) of 0.01-135 ppm deuterium content.

A further aspect is a method for treatment of insulin resistance in which a person requiring treatment is administered with deuterium-depleted food product of 0.01-135 ppm deuterium content.

In all the above cases it is preferred if the deuterium level of the deuterium-depleted water or food is 105-125 ppm.

### DETAILED DESCRIPTION OF THE INVENTION

Development in molecular biology substantially improved our understanding of the mechanism of action of insulin. It is accepted now that the translocation of GLUT proteins (glucose transportases, proteins responsible for glucose transport through the membrane) from the cytoplasm to the membrane on action of insulin is a crucial step in cellular glucose uptake (TRENDS in Biochemical Sciences Vol. 31. No 4 April 2006: Bridging the GAP between insulin signaling and GLUT4 translocation). It was also revealed, however, that GLUT4 (one of the four known glucose transport proteins) showed an insulin-independent translocation, inducible by numerous compounds (nitric oxide, phorbol esters, β- and α-adrenergic antagonists etc.: J. Membrane Biol. 190, 167-174, 2002: Signals that Regulates GLUT4 Translocation). One of the reasons of the insulin resistance in type II DM can be, as supposed by the researchers, that the signal transfer between the insulin receptor and the GLUT4 protein is broken.

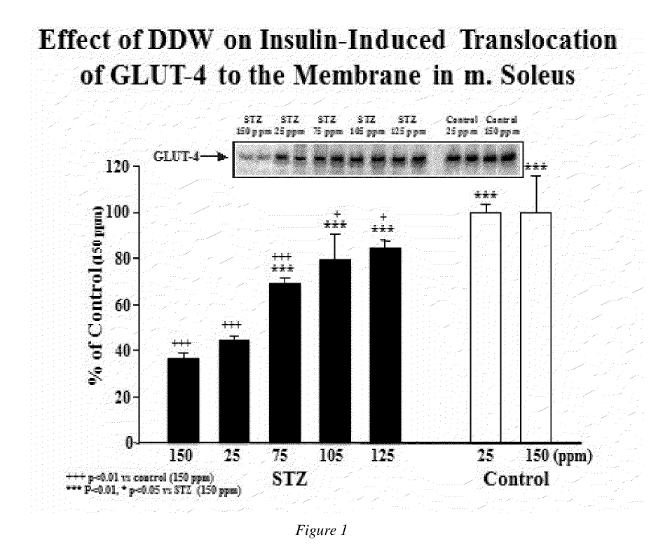

The aim of our last years' research was to reveal the molecular mechanism of the processes induced by lowered D concentration. The effect of D depletion on GLUT4 was investigated by measuring blood sugar levels in streptozotocin (STZ) treated rats lacking insulin production. The interaction of water with various D concentrations (25, 75, 105 and 125 ppm) and insulin dosage (2×1 IU daily) was studied. Blood sugar levels clearly verified the earlier observations about blood glucose decrease on consumption of DDW (28 mmol/L in the group consuming 25 ppm DDW; 50 mmol/L in that consuming 150 ppm-norma-water; p<0.05). The changes in GLUT4 amount are demonstrated in FIG. 1.

Consequences drawn from the results:
1) In healthy, not STZ-treated rats there was no difference between animals consuming 25 ppm or 150 ppm water (two bars on the right). DDW had no effect on the amount of GLUT4 in the membrane.
2) In STZ-treated rats consuming normal water (leftmost bar) the amount of GLUT4 was less than 40% of the control.
3) In STZ-treated rats consuming DDW (25, 75, 105, 125 ppm) GLUT4 amount was higher than in the STZ-control (150 ppm) group.
4) Clear dose-dependence was seen, DDW of 105 and 125 ppm had the strongest effect.

The surprising result of this study was thus that, in the diabetic rat as a model system, D depletion acted primarily not on the insulin receptor but on the number of glucose transporter molecules in the cell membrane, and this is how it enabled the uptake of glucose from the circulating blood. The rats consuming DDW had finally significantly lower blood glucose level. There was a marked contrast to the anti-cancer effect of DDW where lower deuterium levels had stronger tumor inhibitory effect: on the GLUT4 translocation, near-natural deuterium levels (105 and 125 ppm) had the best effect.

According to the above, the invention relates to the use of deuterium depleted water (DDW) for the treatment of insulin resistance based on the effect that DDW can increase the number of GLUT4 (glucose transportase) copies in the membrane, and can so reduce or abolish the cells' insulin resistance, enable normal glucose uptake, and lower blood sugar level.

The invention is based on the recognition that decreasing the normal D concentration in the organism has advantageous influence on glucose metabolism by activating glucose transportase proteins. Their number in the membrane is significantly increased, which stabilizes the unstable blood sugar level at a normal or near-normal level and reduces hyperglycemia. This inventive recognition indicates that lowered D level abolishes insulin resistance—either by restoring the signal pathway between insulin receptors and GLUT proteins or by activating GLUT proteins independently of the signal transduction—and simultaneously normalizes blood glucose.

It is known that beside the daily consumed water volume of 1.2-1.5 L, metabolic processes in the organism generate 0.2-0.3 L of so-called oxidation water from the degradation of organic compounds. To avoid that the D content of oxidation water, originating from organic compounds generated under natural conditions, spoils the effect of the consumed DDW, the invention is further based on the idea that the consumption of organic compounds (carbohydrates, amino acids, lipids) with reduced D content is another way to lower D level in the diseased organism and so to reduce or eliminate insulin resistance, to stabilize blood glucose level and to diminish hyperglycemia.

Accordingly, in the use based on the invention, water with 0.01-135 ppm D (0.021-287 mg/L HDO) and/or carbohydrates, amino acids and lipids with reduced (0.01-135 ppm D) deuterium content, are applied for manufacturing pharmaceutical and food products being suitable for treatment of insulin resistance. Our experiments showed that, within the mentioned interval, 105 and 125 ppm D (that is, 220-262 mg/L HDO) is advantageous.

According to the invention the D content of the water is lowered by a known method, practically by electrolysis or distillation, to 0.01-135 ppm (0.021-287 mg/L HDO), and the water of 0.01-135 ppm D content (0.021-287 mg/L HDO) is used in production of carbohydrates, amino acids and lipids with reduced D content. DDW and the carbohydrates, amino acids and lipids produced by its use are processed by standard pharmaceutical technologies to pharmaceutical products (using the usual vehicles and additives), or by standard food industry technologies to food products. A preferred application for the production of carbohydrates, amino acids and lipids with reduced D content is when water of 0.01-135 ppm deuterium content (0.021-287 mg/L HDO) is used in growing plants and raising animals.

From the methods to produce DDW, electrolysis and distillation are mentioned here in extra because these can yield high amounts of DDW at relatively low costs.
a/ Aqueous KOH solution of 15-20% is electrolysed with 2-5 V DC on separated anode and cathode. Hydrogen with reduced deuterium content, deposited at the cathode, is burnt and the water vapor obtained is condensed in a distillation system and collected separately. The water obtained contains 30-40 ppm deuterium (Separation of Hydrogen Isotopes Eds.: Howard K. Rae, American Chemical Society Symposium Series 68, Washington D.C. 1978; Isotope Separation Eds.: Stelio Villani, American Nuclear Society 1983). By repeated electrolysis, the deuterium content of the water can be further reduced.
b/ Distilled water is boiled in a distillation column with plate number of 50-150, suitable for fractionation, at 50-60 mbar pressure and 45-50° C. Distillation runs at reflux rate of 12-13 and 10-fold bottoms return. With such parameters, the D content of the overhead product is 0.1 to 30 ppm (Separation of Hydrogen Isotopes, Eds.: Howard K. Rae, American Chemical Society Symposium Series 68, Washington D.C. 1978; Isotope Separation Eds.: Stelio Villani, American Nuclear Society 1983). By altering the operation parameters of the column, e.g. by substantially increasing the load, water with D content above 30 ppm can also be produced in large amounts. And, by separating the DDW yielded by the first column on further column(s), deuterium depletion can be increased.

DDW is used as base material in producing the preparations suitable for treatment and curation of diabetes.

The product, manufactured according to the invention, can be used to treat diabetic patients. This is based on the fact that by administering solutions made using DDW, or carbohydrates, amino acids and lipids with reduced deuterium content, D level in the organism will be lowered, resulting in induction of glucose transportase and thus in diminished or abolished insulin resistance and normalized (neither instable nor elevated) blood sugar.

To summarize the results of the basic research work done up to now we can say that in the model experiments consumption of DDW lowered the levels of blood glucose, fructosamine, and hBA1C (where the latter two values indicate blood glucose of the last few weeks), and significantly increased the number of glucose transportase molecules in the membrane. Human experiences of the last years confirmed that consumption of DDW has advantageous effect on the blood sugar level in diabetics. The recognition of DDW consumption on GLUT4 enables the use of deuterium depleting preparations for diminishing or eliminating insulin resistance in diabetic patients.

Products based on the invention can be used in medical practice in forms containing the active component and inert, non-toxic vehicles. The active agent can be processed to products for oral (solution, emulsion, suspension etc.) or parenteral (infusion solution etc.) administration.

Manufacturing of the pharmaceutical products is done by standard methods of this field, by mixing the active agent with inert inorganic or organic vehicles, and preparing galenic formulation from the mixture. A practical vehicle is water.

The pharmaceutical products may contain also other auxiliary components (such as wetting, sweetening or aromatic agents, buffer solutions) usually applied in pharmaceutical industry.

The daily dose of the pharmaceutical products based on the invention can be variable and depends on several factors such as the D concentration of the water, the age and body weight of the patient, the type and severity of diabetes etc. For a 70 kg patient, the daily oral dose can be 0.01-2 L

DDW with 0.01-135 ppm D concentration. To boost both sensory properties and biological effect, the water can contain e.g. 20-30 g/L of D-depleted carbohydrates, certain D-depleted amino acids, or other flavors and aromas.

The advantages of the preparation and process based on the invention are as follows:
a/ Its use enables the increase the number of glucose transporter molecules in the cell membrane and so the reduction or elimination of insulin resistance.
b/ It allows regulation of the patients' blood sugar level.
c/ The use of conventional diabetes medication can be reduced or omitted.
d/ It ensures blood glucose levels within the physiological range, reducing this way the chance of early and late complications.
e/ The chemicals used in the process are non-toxic and non-immunogenic.
f/ Manufacturing the preparations is simple and generates no hazardous waste.

### EXAMPLES

The invention is exemplified with more details, without limiting the scope of protection, hereunder:

A) Pharmacological Example

In the rat model system, animals pretreated with streptozotocin (STZ) were treated with water having various deuterium concentrations for 4 weeks in several independent experiments. The rats had daily two insulin treatments with 1 IU, the control group consumed normal water (150 ppm) while the treated groups consumed DDW of 25, 70, 105, 125, 130, 135, 140 and 145 ppm D level. To find out what mechanism is responsible for the lower blood sugar levels found in the rats drinking DDW, the amount of GLUT4 protein in the rats' muscle cell membrane was determined The rightmost two empty bars in FIG. 1 show that in rats not pretreated with STZ the amount of GLUT4 was not influenced by the D concentration (25-150 ppm) of the water they consumed. The five dark bars on the left side of the figure show that, among the STZ-pretreated rats, the lowest GLUT4 level was in the animals drinking normal water (150 ppm) while in rats drinking DDW it was higher and was maximal at 105 and 125 ppm D level. In accordance with that, the rats' blood sugar was also the lowest in this range (105-125 ppm).

B) Examples of Formulation

Formulation Example 1: Manufacturing of Drinking Water with Advantageous Mineral Composition

D-depleted water and a mineral water of known composition (such as “Csillaghegyi” or “Balfi”) is mixed at the following proportions:
a/ 0.25 parts by volume of 90 ppm DDW+0.75 parts mineral water (final D concentration: 135 ppm);
b/ 0.5 parts by volume of 90 ppm DDW+0.5 parts mineral water (final D concentration: 120 ppm);
c/ 0.75 parts by volume of 90 ppm DDW+0.25 parts mineral water (final D concentration: 105 ppm);
d/ 0.25 parts by volume of 60 ppm DDW+0.75 parts mineral water (final D concentration: 127.5 ppm);
e/ 0.5 parts by volume of 60 ppm DDW+0.5 parts mineral water (final D concentration: 105 ppm);

Formulation Example 2: Cation and Anion Content of DDW is Set by an Artificial Concentrate of Advantageous Salt Composition.

A possible composition of the stock solution is as follows:

| | |
|---|---|
| KCl | 5.7 g |
| MgCl₂ × 6 H₂O | 199.65 g |
| CaCl₂ × 6 H₂O | 236.25 g |

By adding this stock solution to 1,000 L of DDW, the final concentrations will be (in mg/L): Mg²⁺, 23.8; Ca²⁺, 64.1; K⁺, 3; Cl⁻, 192.

Formulation Example 3: Manufacturing Food Products with Reduced D Content

Green peppers (paprika), tomatoes, green peas, French beans etc. are grown by standard gardening methods, using water of 0.01-135 ppm D content. The crop is processed to food products by routine procedures of food industry.

Formulation Example 4: Production of Deuterium-Depleted Carbohydrates (Sugars)

Water containing 0.01-135 ppm D (0.021-287 mg/L HDO) is used for irrigation in growing sugar beats—being rich in sugar—under greenhouse conditions. Sugar is extracted from the plants grown with DDW by the general methods of sugar beet processing.

Formulation Example 5: Production of Deuterium-Depleted Proteins

Water containing 0.01-135 ppm D (0.021-287 mg/L HDO) is used for irrigation in growing soy beans—being rich in proteins—under greenhouse conditions. The plants grown with DDW are processed to human food and animal feed by the usual methods of the corresponding industrial branch.

Formulation Example 6: Production of Deuterium-Depleted Lipids/Oils

Water containing 0.01-135 ppm D (0.021-287 mg/L HDO) is used for irrigation in growing sunflower—being rich in vegetable oil—under greenhouse conditions. The plants grown with DDW are processed by the usual methods of food and feed industry.

Formulation Example 7: Production of Deuterium-Depleted Food Rich in Proteins and Lipids

Plants grown by using water containing 0.01-135 ppm D (0.021-287 mg/L HDO) for irrigation are processed by standard methods to animal feed. This deuterium-depleted feed is given to farm animals whose drinking water is replaced by water containing 0.01-135 ppm D (0.021-287 mg/L HDO). The animals are slaughtered and processed by standard methods.

1. Deuterium depleted water (DDW) of 0.01-135 ppm deuterium concentration for use in the treatment of insulin resistance.

2. A pharmaceutical product applicable in the treatment of insulin resistance comprising the deuterium depleted water (DDW) of 0.01-135 ppm deuterium concentration of claim 1.

3. The deuterium depleted water according to claim 1, where the deuterium content of the water is 105-125 ppm.

4. Deuterium depleted food product of 0.01-135 ppm deuterium concentration for use in the treatment of insulin resistance.

5. Use of deuterium depleted water (DDW) of 0.01-135 ppm deuterium concentration of claim 1 in manufacturing of food products applicable in treatment of insulin resistance.

6. The food product according to claim 4, where the deuterium content of the food product is 105-125 ppm.

7. The food product according to claim 4, where the food product contains carbohydrates, proteins and lipids with a deuterium content of 105-125 ppm.

8. A method for the treatment of insulin resistance, said method comprising administering to a person requiring the treatment deuterium depleted water of 0.01-135 ppm deuterium content or deuterium depleted food product of 0.01-135 ppm deuterium content.

9. The method for the treatment of insulin resistance of claim 8, comprising administering to a person requiring the treatment deuterium depleted water of 0.01-135 ppm deuterium content.

10. The method according to claim 9, where the deuterium content of the water is 105-125 ppm.

11. The method for the treatment of insulin resistance of claim 8, comprising administering to a person requiring the treatment deuterium depleted food product of 0.01-135 ppm deuterium content.

12. The method according to claim 11, where the deuterium content of the food product is 105-125 ppm.

13. The method according to claim 11, where the food product contains carbohydrates, proteins and lipids with a deuterium content of 105-125 ppm.
